(19) United States
(12) Patent Application Publication
(10) Pub. No.: US 2023/0055178 A1
(43) Pub. Date: Feb. 23, 2023

(54) CYLINDRICAL EMBEDDED MATERIAL FOR MEDICAL USE

(71) Applicant: HI-LEX CORPORATION, Takarazuka-shi, Hyogo (JP)

(72) Inventor: Shinsaku Koarashi, Takarazuka-shi, Hyogo (JP)

(21) Appl. No.: 17/792,488

(22) PCT Filed: Jan. 29, 2021
(86) PCT No.: PCT/JP2021/003130

§ 371 Date: Jul. 13, 2022

(30) Foreign Application Priority Data
Jan. 30, 2020 (JP) 2020-014011

(51) Int. Cl. A61F 2/06; A61L 31/06

(52) U.S. Cl. CPC A61F 2/06; A61L 31/06; A61F 2220/0075

(57) ABSTRACT

Provided is a cylindrical embedded material for medical use, in which a plurality of structure bodies are connected in an axial direction, wherein the structure body elements each have an enclosure part configured for enclosing inside thereof, wherein the cylindrical embedded material connection part in which a structure body element of a first structure body and a structure body element of a second structure body connected in the axial direction, wherein the enclosure part has a spacing part to separate the fibers, wherein the cylindrical embedded material has a covering part, and wherein the covering part has elasticity which allows for movement of the fibers to narrow the spacing part when an external force is applied to the structure body element and allows for restoration of positions of the moved fibers when the external force is removed.

### TECHNICAL FIELD

The present invention relates to a cylindrical embedded material for medical use such as an artificial blood vessel.

### BACKGROUND ART

Conventionally, an artificial blood vessel (graft) to be embedded into a human body, which is one example of a cylindrical embedded material for medical use, is formed of synthetic fibers such as polyester with a woven structure or a knitted structure.

The knitting structure (also referred to as a knitted fabric) is formed by creating a loop with threads and entwining subsequent threads with the loop to form a continuous loop, thereby connecting them to each other. Since the artificial blood vessel with the knitted structure has a high porosity, the blood leakage resistance performance thereof is low.

Therefore, an artificial blood vessel coated with urethane elastomer on the entire outer peripheral surface has been used. However, the porosity of the artificial blood vessel is easily impaired by a coating layer of urethane elastomer, and the entire outer peripheral surface is covered with urethane elastomer, thereby making it difficult to selectively puncture a needle in a hole provided between fibers. Therefore, when a needle hole is formed in the coating layer by puncturing with a suture needle or a syringe, a needle hole in which a yarn is cut by the edge of the needle tip is easily generated and the needle hole is likely to remain. As a means for achieving both porosity property that is such high porosity and blood leakage prevention, for example, Patent Document 1 discloses a blood vessel repairing material in which a yarn is welded at a part of yarn joint portion at which a texture or a seam is formed.

### PRIOR ART DOCUMENT

### Patent Document

Patent Document 1: JP H04-003982 B2

### SUMMARY OF THE INVENTION

### Problem to be Solved by the Invention

However, when the blood vessel repairing material of Patent Document 1 is used as an artificial blood vessel, the holes provided between the fibers still exist, and therefore, the needle tip becomes easy to enter the holes, but the needle hole after the punctured needle being pulled out can be maintained in an open state due to the entanglement of the fibers, which makes it difficult to improve the blood leakage resistance performance.

It is an object of the present invention to provide a cylindrical embedded material for medical use which is excellent in blood leakage resistance performance.

### Means to Solve the Problem

The cylindrical embedded material for medical use of the present invention is:

a cylindrical embedded material for medical use, in which a plurality of structure bodies, each configured so that a plurality of structure body elements are arranged around an axis, are connected in an axial direction,

wherein the structure body elements each have an enclosure part configured for enclosing inside thereof with yarn in which a plurality of fibers are bundled,

wherein the cylindrical embedded material for medical use has a connection part in which a structure body element of a first structure body and a structure body element of a second structure body adjacent to the first structure body are connected in the axial direction,

wherein the enclosure part has a spacing part to separate the fibers so as to have a spread in a direction around the axis of the structure body,

wherein the cylindrical embedded material for medical use has a covering part covering the enclosure part and the connection part with resin, and

wherein the covering part has elasticity which allows for movement of the fibers to narrow the spacing part when an external force is applied to the structure body element and allows for restoration of positions of the moved fibers when the external force is removed.

### Effects of the Invention

According to the present invention, a cylindrical embedded material for medical use can be realized which is excellent in blood leakage resistance performance.

### BRIEF DESCRIPTION OF THE DRAWINGS

FIG. 1 is an outline view schematically showing a cylindrical embedded material for medical use according to an embodiment of the present invention.

FIG. 2 is a partially enlarged view of an outer surface of the cylindrical embedded material for medical use according to an embodiment of the present invention.

FIG. 3 is a partially enlarged view of an inner surface of the cylindrical embedded material for medical use according to an embodiment of the present invention.

FIG. 4 is a partially enlarged view schematically showing an outer surface of the cylindrical embedded material for medical use according to an embodiment of the present invention.

FIG. 5 is a view showing a state where a needle is punctured onto an outer surface of the cylindrical embedded material for medical use according to an embodiment of the present invention.

FIG. 6 is a view showing one example of a structure of the cylindrical embedded material for medical use according to an embodiment of the present invention.

### EMBODIMENT FOR CARRYING OUT THE INVENTION

Hereinafter, embodiments of the present invention will be described in detail with reference to the drawings.

### <Movement of Cylindrical Embedded Material for Medical Use 1>

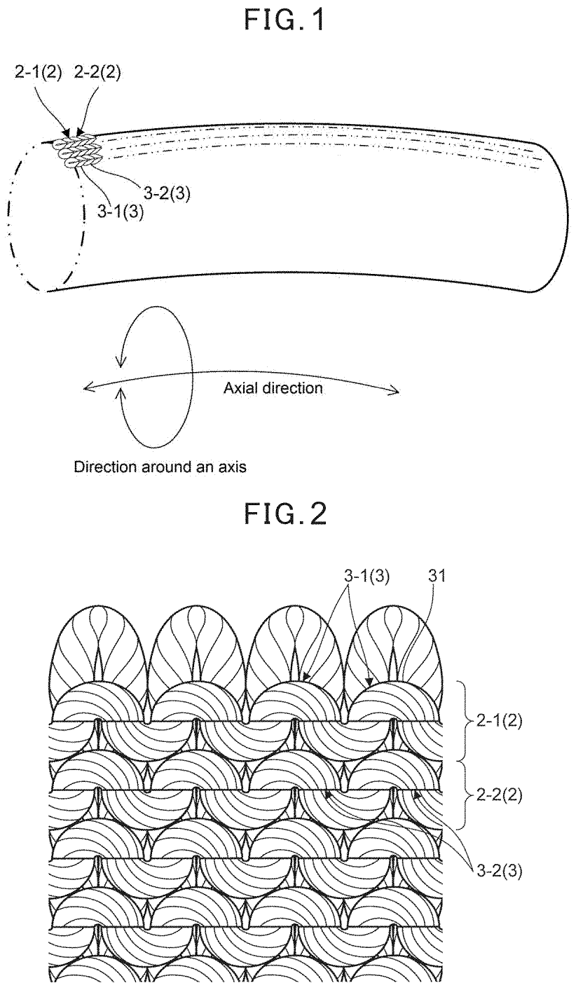

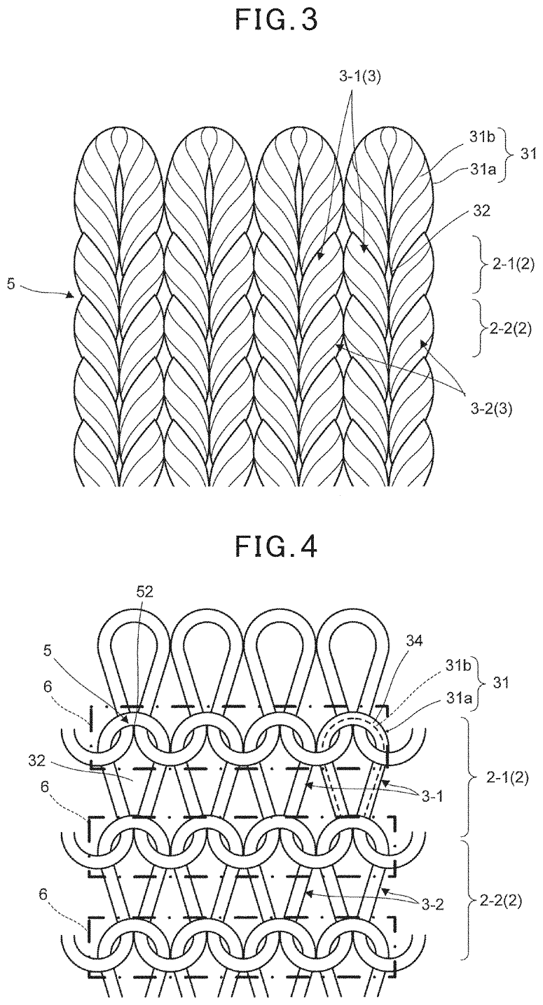

The cylindrical embedded material for medical use 1 shown in FIGS. 1 to 3 is a tubular member that is embedded and used in a living body such as a human body. The cylindrical embedded material for medical use 1 can be used in place of a biological tube including a digestive tract such as an esophagus, or can be used as a shunt. In this embodiment, a case where the cylindrical embedded material for medical use 1 is applied to an artificial blood vessel including a stent graft will be described.

The cylindrical embedded material for medical use 1 is made of fibers. In this embodiment, the cylindrical embedded material for medical use 1 is formed into a cylindrical shape as a whole by weaving yarn. The yarn is made of a bundle of multiple fibers.

In the cylindrical embedded material for medical use 1 of this embodiment, the yarn is formed in a loop shape, forming a structure body element 3. The structure body elements 3 are continuously formed in a direction around an axis of the cylindrical embedded material for medical use 1, constituting a first structure body 2-1. Moreover, the structure body elements 3 are continuously formed in the direction around the axis at positions adjacent to the first structure body 2-1, constituting a second structure body 2-2. Here, the structure body element(s) 3 constituting the first structure body 2-1 are referred to as first structure body element(s) 3-1 in the following description, and the structure body element(s) 3 constituting the second structure body 2-2 are referred to as second structure body element(s) 3-2 in the following description. Each of the second structure body elements 3-2 is woven with respect to each of the first structure body elements 3-1 arranged in the direction around the axis so that yarn in the second structure body element 3-2 is looped and hooked to each of the first structure body elements 3-1. The second structure body 2-2 and the first structure body 2-1 have structures coupled to each other by being entangled with each other. The second structure body 2-2 is connected to the first structure body 2-1 in an axial direction. The cylindrical embedded material for medical use 1 of this embodiment has a so-called stockinette stitch structure.

In the cylindrical embedded material for medical use 1, a plurality of structure bodies 2, each configured so that a plurality of structure body elements 3 are arranged around the axis, are connected in the axial direction.

The structure body elements 3 form annular structure body 2 by being continuously arranged in the direction around the axis. In this embodiment, structure bodies 2 each formed in an annular shape by a plurality of structure body elements 3 are provided continuously in the axial direction, in to the cylindrical embedded material for medical use 1.

The plurality of structure body elements 3 are formed by yarn 31.

As the yarn 31, fibers such as, for example, polyester, polyamide and urethane are used, which are configured by bundling a plurality of synthetic fibers 31a, 31b together.

The structure body elements 3 each have an enclosure part 32 configured to enclose the inside so that the yarn 31 forms a predetermined inner space. Inside the enclosure part 32, the enclosure part 32 may include fibers or have a space.

Each of the plurality of fibers constituting the yarn surrounding the enclosure part 32 spreads in the direction around the axis, and is arranged at an interval that can prevent liquid from leaking from the inside of the cylindrical embedded material for medical use 1. That is, the enclosure part 32 has a gap so that the fibers are movable, although a space in the enclosure part 32 is closed by each of the plurality of fibers of the yarn 31 surrounding thereof, which makes it difficult for liquid to leak from the enclosure part 32. FIG. 6 shows one example of a state where fibers of the yarn spread in the direction around the axis on the outer surface of the cylindrical embedded material for medical use 1.

Here, with reference to FIG. 4, a connection relationship between the structure body elements 3 of each structure body 2 in the cylindrical embedded material for medical use 1 will be described. FIG. 4 is a partially enlarged view schematically showing the outer surface of the cylindrical embedded material for medical use, the view schematically showing a connection between yarn of the structure body elements 3 shown in FIG. 2.

In the structure body 2, the first structure body element 3-1 and the second structure body element 3-2 in the first structure body 2-1 and the second structure body 2-2 that are adjacent to each other in the axial direction are connected to each other at a connection part 5 without being welded to each other.

The connection part 5 is a part where the first structure body element 3-1 and the second structure body element 3-2 in the first structure body 2-1 and the second structure body 2-2 that are adjacent to each other in the axial direction come into contact with each other. The connection part 5 is covered by a covering part 6 formed by resin entering a contacting part where the structure body element 3-1 and the structure body element 3-2 come into contact.

The enclosure part 32 has a spacing part 34 to separate the fibers (in FIG. 4, for convenience, among the plurality of synthetic fibers, the synthetic fibers 31a and 31b are schematically shown) so as to have a spread in a direction around the axis of the structure body 2. The enclosure part 32 of this embodiment is an inner part of a loop formed by the yarn 31, the loop constituting the structure body element 3.

Movements of the plurality of fibers of the yarn 31 constituting the enclosure part 32 are restricted by the covering part 6 at the base of the enclosure part 32, that is, the root of the loop.

The covering part 6 covers the enclosure part 32 and the connection part 5. The covering part 6 is formed of resin such as urethane elastomer.

The covering part 6 covers the plurality of fibers of the yarn 31 in the enclosure part 32 so as to have a spacing part 34 to separate the fibers in the direction around the axis.

By covering the connection part 5 with the covering part 6, high blood leakage resistance state, in which each of the fibers of the structure body element 3-1 and the structure body element 3-2 spreads in the direction around the axis in state where no external force is applied to the structure body element 3 to close the enclosure part 32, etc., can be obtained.

The covering part 6 has elasticity which allows for movement of the fibers to narrow the spacing part 34 when an external force is applied to the structure body element 3 and allows for restoration of positions of the moved fibers when the external force is removed. The case where the external force is applied to the structure body element 3 is a case where a force for moving a plurality of fibers is applied, such as, for example, a case where a suture needle for suturing when used for an artificial blood vessel or an injection needle such as a syringe is punctured into the cylindrical embedded material for medical use 1.

The covering part 6 has rigidity for maintaining a state in which the fibers spread in the direction around the axis of the structure body 2 by the spacing part 34 when no external force is applied. Therefore, the covering part 6 constitutes a high blood leakage resistance structure in which body fluid, for example, blood does not leak from the inside to the outside of the cylindrical embedded material for medical use 1.

In this embodiment, the covering part 6 is provided at an intersection part 52 where the yarn 31 of the structure body elements 3 intersect. Therefore, even if fibers led out from the intersection part 52 are displaced by an external force, the displaced fibers return to their original positions when the external force is removed.

The covering part 6 covers the connection part 5, i.e., the intersection part 52 of the yarn 31, with resin, and connects the structure body element 3-1 of the first structure body 2-1 and the structure body element 3-2 of the second structure body 2-2 adjacent to the first structure body 2-1, thereby connecting a plurality of structure bodies 2. The covering part 6 holds fibers of respective yarn 31 of the structure body element 3-1 of the first structure body 2-1 and the structure body element 3-2 of the second structure body 2-2 in a state where a plurality of fibers of the yarn 31 are separated by the spacing part 34 so as to have a spread in the direction around the axis.

Since the covering part 6 covers the connection part 5 with the resin, even when the cylindrical embedded material for medical use 1 is cut, for example, when the cylindrical embedded material for medical use 1 having a predetermined length is formed by the cutting, the yarn is not unraveled on the cutting surface and appropriately returns to its original position even when the site is stretched. Moreover, in case that a needle having a diameter smaller than the connection part 5 is punctured into the covering part 6 during suturing or treatment, the covering part 6 may be configured to cover the enclosure part 32 and the connection part 5 so that the covering part 6 has an outer surface to guide the needle inside the enclosure part 32 or between the enclosure parts 32.

### <Movement of Cylindrical Embedded Material for Medical Use 1>

In the cylindrical embedded material for medical use 1, a plurality of structure body elements 3 each having an enclosure part 32 are continuously arranged in an axial direction and a direction around an axis. The structure body elements 3 are connected at the connection part 5 being a stitch, in a state of being adjacent to each other in the axial direction. The connection parts 5 are covered with the covering part 6.

In a normal state of the cylindrical embedded material for medical use 1, the yarn constituting each structure body 2 and each structure body element 3 is in a state where a gap part including the enclosure part 32 is closed in the direction around the axis due to the spread of the plurality of fibers in the direction around the axis.

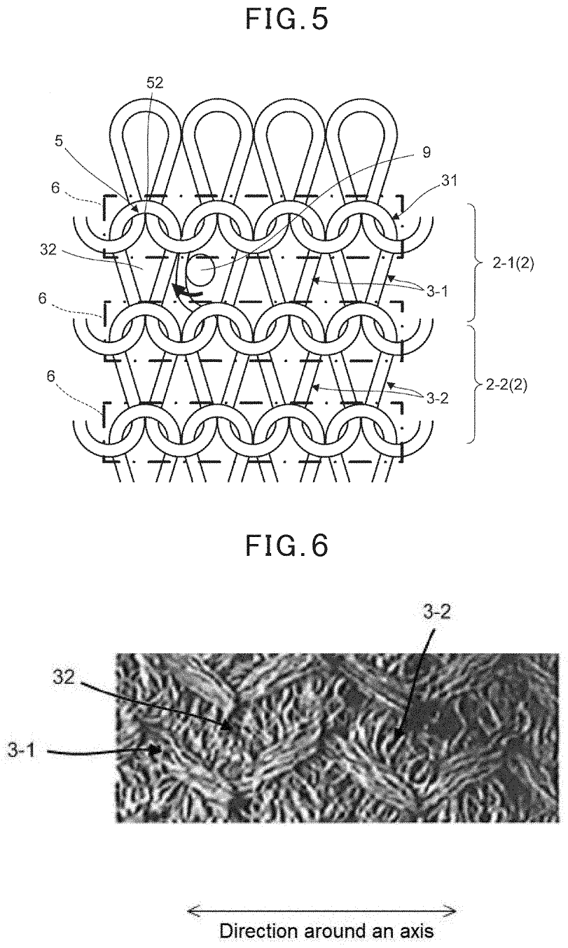

As shown in FIG. 5, when a needle 9 is punctured into the cylindrical embedded material for medical use 1 as an external force to the cylindrical embedded material for medical use 1, the needle 9 abuts the yarn 31 of the structure body element 3, or guided between the yarn or between the fibers of the yarn 31, for example, into the enclosure part 32.

When the needle 9 is punctured between the fibers of the yarn such as the enclosure part 32, the puncturing force is transmitted to the fiber in contact with the needle 9 via the needle 9, and this fiber is pushed by the needle 9 to be displaced (see the arrow in FIG. 5). Moreover, this displaced fiber further pushes the adjacent fiber to be displaced.

That is, in the enclosure part 32 surrounded by a bundle of fibers, the spacing part 34 becomes narrower, the punctured site becomes a needle hole, and the needle 9 is in a state being inserted into this needle hole. The needle 9 is thus inserted into the cylindrical embedded material for medical use 1 against a biasing force of the covering part 6 that covers the enclosure part 32.

Then, when the needle 9 comes out of the needle hole and the external force on the structure body 2 is removed, the fiber returns to its original position due to the biasing force of the covering part 6, and the part where the needle hole is formed turns into a state such that it is covered with the originally existing fibers, maintaining the blood leakage resistance.

Furthermore, the covering part 6 may be formed in a shape to guide the needle to the enclosure part 32 when the needle 9 abuts the covering part 6. In this case, in the cylindrical embedded material for medical use 1, a suitable suturing can be performed without the needle 9 being guided to a site where it is not desired to be punctured.

### <Producing Method>

By knitting yarn of synthetic resin, a cylindrical embedded material body in which a plurality of structure bodies 2, each configured so that a plurality of structure body elements 3 are arranged around an axis, are connected in an axial direction is produced. The produced cylindrical embedded material body is immersed in a solution obtained by diluting resin which forms a covering part 6, for example, urethane elastomer with water alcohol for a predetermined time and then pulled up.

In this way, urethane elastomer is stored in a part where fibers intersect with each other, that is, a connection part 5 in the embodiment, rather than in parts where the yarn (the fibers in the yarn) surrounding an enclosure part 32, etc., do not intersect with each other, due to a capillary phenomenon. Although urethane elastomer is applied to parts where a small number of fibers are adjacent to each other, urethane elastomer is stored in the connection part 5 where structure body elements 3-1 and 3-2 are connected in the axial direction, in other words, where the fibers in the yarn intersect with each other.

By drying the cylindrical embedded material body in this state, the structure body elements 3-1 and 3-2 of the cylindrical embedded material body are fixed in a state where each fiber of the yarn of the structure body elements 3-1 and 3-2 is spread in the direction around the axis by the covering part 6, which is urethane elastomer. As a result, a cylindrical embedded material for medical use 1 is produced and each fiber of the yarn becomes movably fixed in a state such that it does not leak liquid inside to the outside.

According to this embodiment, even if an external force is applied the structure body element 3 and the fibers move to narrow the spacing part 34 of the fibers in the enclosure part 32, the positions of the moved fibers can be restored by elasticity of the covering part 6 when the external force is removed. Before moving, the fibers constituting the spacing part 34 are separated from each other, have a spread in the direction around the axis of the structure body, and the spacing parts 34 are arranged so as not to have a gap with each other around the axis, so that the structure body can be in a state of a high blood leakage resistance. Therefore, even if a needle hole is formed such as by puncture, it can be self-repaired by moving the fibers and closed, and the structure body can be maintained in a form with the high blood leakage resistance performance.

The cylindrical embedded material for medical use 1 of this embodiment is configured by knitting yarn of synthetic resin by stockinette stitch, but the present invention is not limited to this configuration, and it may be formed by any knitting methods having the above-described configuration.

The embodiments of the present invention have been described above. Besides, the descriptions above are illustrative of preferred embodiments of the present invention, and the scope of the present invention is not limited thereto. That is, the descriptions of the configurations of the cylindrical embedded material for medical use and the shape of each part are merely examples, and it is obvious that various changes and additions to these examples are possible within the scope of the present invention.

### INDUSTRIAL APPLICABILITY

The cylindrical embedded material for medical use according to the present invention has an excellent blood leakage resistance performance, has an effect that it is easy to handle, and is useful as a material applied to stent grafts, artificial blood vessels, and the like.

### REFERENCE SIGNS LIST

1. Cylindrical embedded material for medical use
2. Structure body
3. Structure body element
5. Connection part
6. Covering part
32. Enclosure part
34. Spacing part

1. A cylindrical embedded material for medical use, in which a plurality of structure bodies, each configured so that a plurality of structure body elements are arranged around an axis, are connected in an axial direction,
wherein the structure body elements each have an enclosure part configured for enclosing inside thereof with yarn in which a plurality of fibers are bundled,
wherein the cylindrical embedded material for medical use has a connection part in which a structure body element of a first structure body and a structure body element of a second structure body adjacent to the first structure body are connected in the axial direction,
wherein the enclosure part has a spacing part to separate the fibers so as to have a spread in a direction around the axis of the structure body,
wherein the cylindrical embedded material for medical use has a covering part covering the enclosure part and the connection part with resin, and
wherein the covering part has elasticity which allows for movement of the fibers to narrow the spacing part when an external force is applied to the structure body element and allows for restoration of positions of the moved fibers when the external force is removed.

2. The cylindrical embedded material for medical use of claim 1, wherein the covering part has rigidity for maintaining a state in which the fibers have a spread in the direction around the axis of the structure body by the spacing part when no external force is applied.

3. The cylindrical embedded material for medical use of claim 1, wherein the resin comprises urethane elastomer.

4. The cylindrical embedded material for medical use of claim 1, wherein the connection part comprises an intersection part where the yarn of respective structure body elements connected with each other intersects, and wherein the covering part is provided at the intersection part.
